(19) United States
(12) Patent Application Publication
(10) Pub. No.: US 2012/0238959 A1
(43) Pub. Date: Sep. 20, 2012

(54) Biased Internal Bolster for a Medical Device

(75) Inventors: David L. Thorne, Kaysville, UT (US); Steven A. Weir, Sandy, UT (US); Andrew Sheffield, Kaysville, UT (US); Amir Orome, Sandy, UT (US); Charles Farnworth, Riverton, UT (US)

(73) Assignee: C.R. Bard, Inc., Murray Hill, NJ (US)

(21) Appl. No.: 13/419,185

(22) Filed: Mar. 13, 2012

Related U.S. Application Data
(60) Provisional application No. 61/452,339, filed on Mar. 14, 2011.

(51) Int. Cl. A61M 25/04

(52) U.S. Cl. 604/177; 604/174

(57) ABSTRACT

A catheter and internal bolster for securing the catheter or other medical device within a body of a patient. In one embodiment, the catheter comprises an elongate catheter tube that defines at least one lumen and an internal bolster disposed proximate a distal end of the catheter tube. The internal bolster includes a plurality of hinged arms and is capable of an undeployed position wherein each arm is longitudinally extended substantially parallel to the catheter tube, and a deployed position wherein each arm is folded about its hinge so as to radially extend from the catheter tube and provide a bolster configuration. The internal bolster further includes a resilient portion included with the catheter tube and disposed centrally with respect to the internal bolster. The resilient portion provides a force urging the internal bolster to the deployed position. In one possible implementation, the catheter is configured as a feeding tube.

### CROSS-REFERENCE TO RELATED APPLICATIONS

This application claims the benefit of U.S. Provisional Patent Application No. 61/452,339, filed Mar. 14, 2011, and titled “Biased Internal Bolster for a Medical Device,” which is incorporated herein by reference in its entirety.

### BRIEF SUMMARY

Briefly summarized, embodiments of the present invention are directed to a catheter and internal bolster for securing the catheter or other medical device within a body of a patient. In one implementation, the catheter is configured as a gastrostomy (“feeding”) tube disposed within a percutaneous stoma defined in the body of the patient, so as to establish a feeding conduit to the stomach of the patient.

In one embodiment, the catheter comprises an elongate catheter tube that defines at least one lumen and an internal bolster disposed proximate a distal end of the catheter tube. The internal bolster includes a plurality of hinged arms and is capable of an undeployed position wherein each arm is longitudinally extended substantially parallel to the catheter tube, and a deployed position wherein each arm is folded about its hinge so as to radially extend from the catheter tube and provide a bolster configuration. The internal bolster further includes a resilient portion included with the catheter tube and disposed centrally with respect to the internal bolster. The resilient portion provides a force urging the internal bolster to the deployed position.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A-1D show various views of a feeding catheter including an internal bolster according to one embodiment;

FIG. 2 is a side view of an obturator for use with the catheter of FIGS. 1A-1D;

FIGS. 3A and 3B show extension of the internal bolster of the catheter of FIGS. 1A-1D using the obturator of FIG. 2, according to one embodiment;

FIG. 4 is a cross-sectional side view of the catheter of FIGS. 1A-1D in place within a stoma;

FIGS. 5A and 5B show various views of an internal bolster according to one embodiment;

FIGS. 6A and 6B show various views of a feeding catheter including an internal bolster according to one embodiment;

FIGS. 7A and 7B show various views of a feeding catheter including an internal bolster according to one embodiment;

FIGS. 8A and 8B show various cross-sectional views of a feeding catheter including an internal bolster according to one embodiment;

FIG. 9 is a cross sectional view of a proximal portion of a feeding catheter including a flushing lumen according to one embodiment;

FIGS. 10A and 10B show various views of a feeding catheter including an internal bolster according to one embodiment; and

FIGS. 11A and 11B show various views of a feeding catheter including an internal bolster according to one embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a feeding device placed within the body of a patient is considered a distal end of the device, while the device end remaining outside the body is a proximal end of the feeding device. Also, the words “including,” “has,” and “having,” as used herein, including the claims, shall have the same meaning as the word “comprising.”

Embodiments of the present invention are generally directed to an internal bolster for securing a catheter or other medical device within a body of a patient. In one implementation, for instance, the catheter is configured as a gastrostomy (“feeding”) tube that is disposed within a percutaneous stoma defined in the body of the patient, so as to establish a conduit to the stomach of the patient.

In accordance with one embodiment, the internal bolster is biased so as to preferentially remain in a deployed configuration so as to prevent inadvertent removal of the bolster from within the patient. The internal bolster can be selectively deformed to an undeployed configuration, via use of an obturator for example, to enable insertion or removal of the medical device from the patient. Once insertion or removal is completed and the obturator removed, the biased internal bolster of the device returns to its deployed configuration. Various configurations are disclosed herein for preferentially maintaining the internal bolster in the deployed configuration.

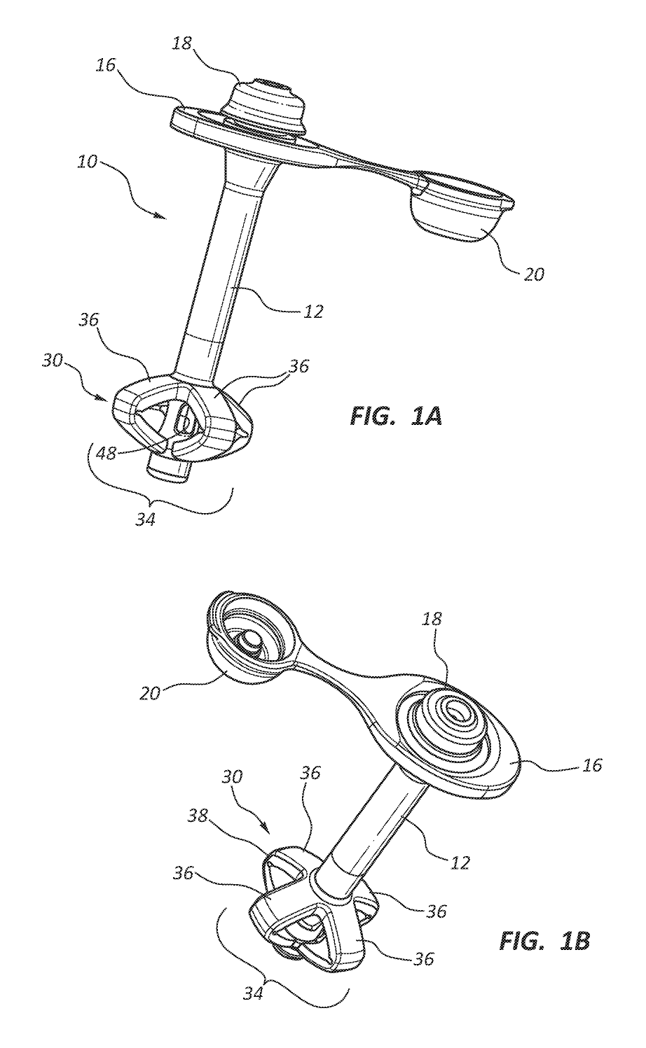

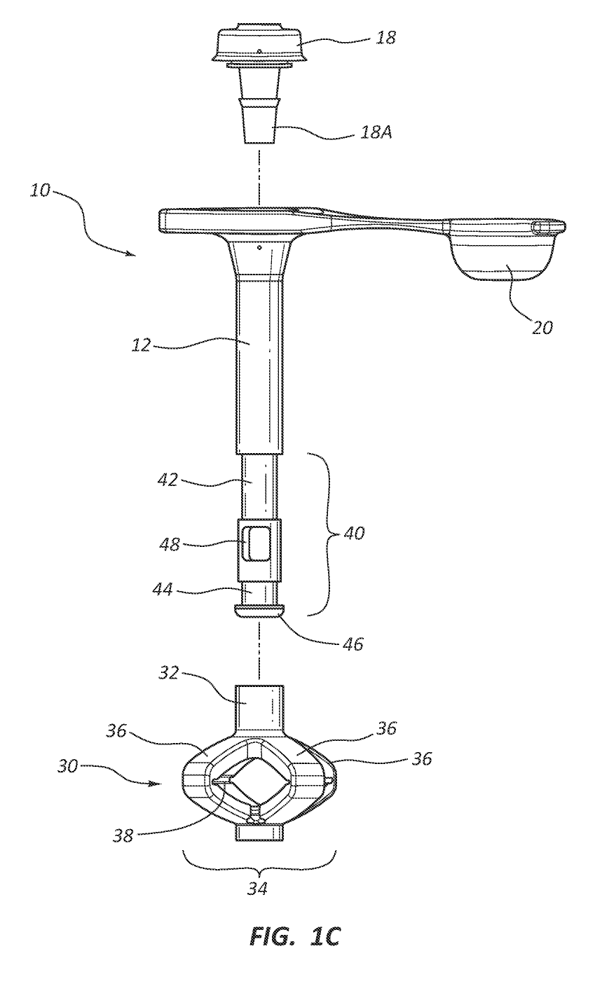

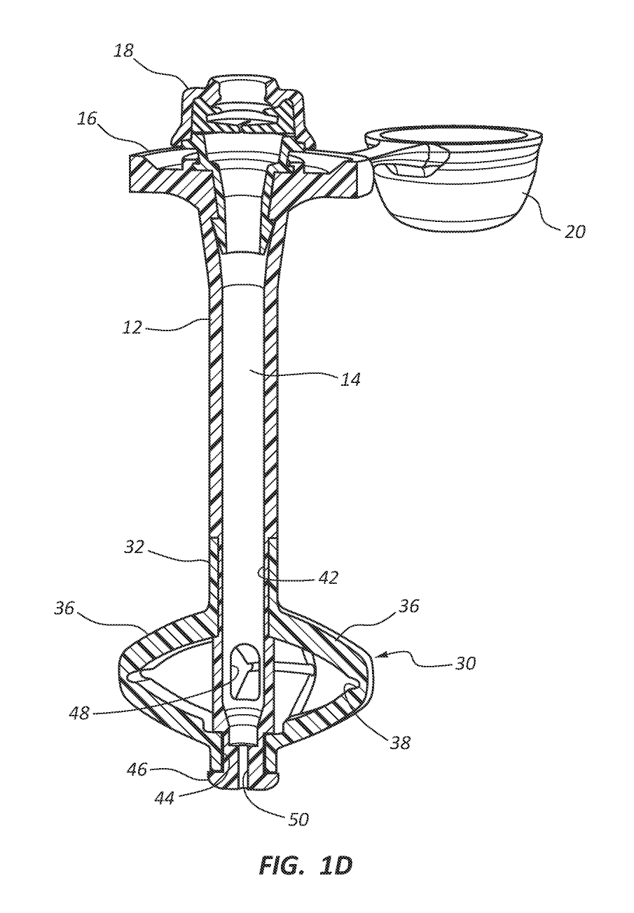

FIGS. 1A-1D show various features of a catheter, generally designated at 10 and configured here as a feeding tube, configured according to one embodiment. The catheter 10 shown here is thus one example of a feeding tube that establishes a conduit via a percutaneous stoma to the stomach of a patient for providing enteral nutrition to the patient.

As shown, the catheter 10 includes an elongate catheter tube 12 as the body of the catheter. The catheter tube 12 defines at least one lumen 14 through which nutritive fluids or other substances may pass. An external bolster 16 is included proximate a proximal end of the tube 12 to provide a stop for the external portion of the catheter 10 when the external bolster is adjacent the skin surface of the patient. A valve assembly 18 including a slit, tricuspid, or other suitable type of valve can be included. The valve assembly 18 includes a neck 18A that is securely received into the proximal end of the catheter tube lumen 14. A cap 20 is included to removably cover the top of the valve assembly 18.

FIGS. 1A-1D further depict details regarding an internal bolster 30, configured according to one embodiment. The bolster 30 is provided proximate a distal end of the catheter tube 12 so as to prevent inadvertent removal of the catheter 10 from the stoma after insertion into the patient. As shown, the bolster 30 includes a hollow body 32 from which extends a plurality, or array 34, of hinged arms 36. Each arm 36 includes a hinge 38 to enable the arm to extend or fold according to the desired use and configuration, as will be shown below. In another embodiment, the arms are flexible but include no hinges.

As best seen in FIG. 1C, the catheter tube 12 further includes a distal portion 40 that extends to a distal end of the catheter tube and defines a distal portion of the lumen 14. The distal portion 40 defines an annular first notch 42 and an annular second notch 44 distal to and spaced apart from the first notch. A lip 46 is included at the distal end of the catheter tube 12 adjacent the second notch 44. An opening 48 in communication with the lumen 14 is also defined by the distal portion of the catheter tube 12 and is disposed between the first notch 42 and the second notch 44.

The first and second notches 42, 44 are sized and spaced so as to receive corresponding portions of the body 32 of the internal bolster 30 when the bolster is mated to the catheter tube 12, as seen in FIG. 1D. Together with the notches 42, 44, the lip 44 is configured to maintain engagement of the internal bolster 30 with the catheter tube 12. As seen in FIG. 1D, the opening 48 is positioned within the array 34 of arms 36 to enable fluids passed through the catheter 10 to exit from the catheter tube 12 and into the stomach of the patient. FIG. 1D further shows that in the present embodiment, the hinge 38 for each arm 36 includes a notch to facilitate folding of the arm, as seen here. A guidewire conduit 50 is also shown in FIG. 1D, extending through the distal end of the catheter tube 12 to enable passage of a guidewire through the catheter tube, if desired during placement thereof into the patient.

As will be seen, the distal portion 40 of the catheter tube 12 is resilient so as to enable stretching thereof. This in turn enables stretching of the flexible internal boslter 30 when it is longitudinally extended, as described below. The resilience of the distal portion 40 also provides an urging force for returning the internal bolster to its rest state with its arms 36 in a folded, radially extended configuration as seen in FIGS. 1A and 1B. In the present embodiment, the entire catheter tube includes a resilient, deformable material such as silicone or other suitable material. In another embodiment, only the distal portion of the catheter tube is resilient. Further it is appreciated that, while it is integrally formed with the catheter tube here, in other embodiments the distal portion can be separately formed and subsequently attached to the catheter tube.

Likewise, though in the present embodiment it is separately manufactured before attachment to the catheter tube, in other embodiments the internal bolster can be manufactured integrally with the catheter tube. These and other variations are therefore contemplated. In the present embodiment the internal bolster 30 includes silicone, though other medically or otherwise suitable materials, including polyurethane and other thermoplastics, can be employed. In one embodiment, the internal bolster 30 includes silicone of 70 Shore A durometer. Of course, other suitable materials and durometers can be employed.

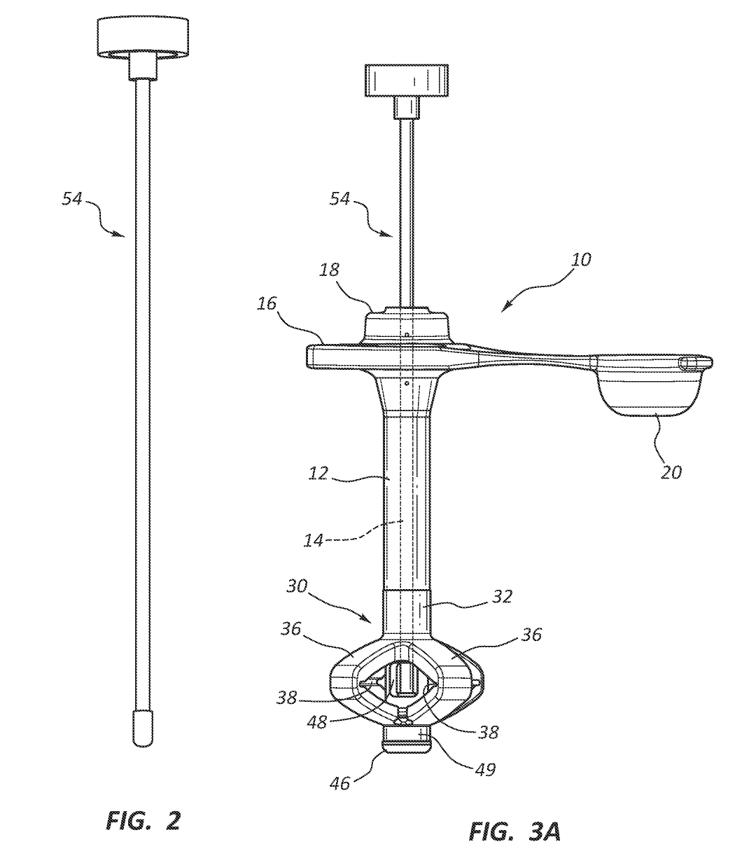

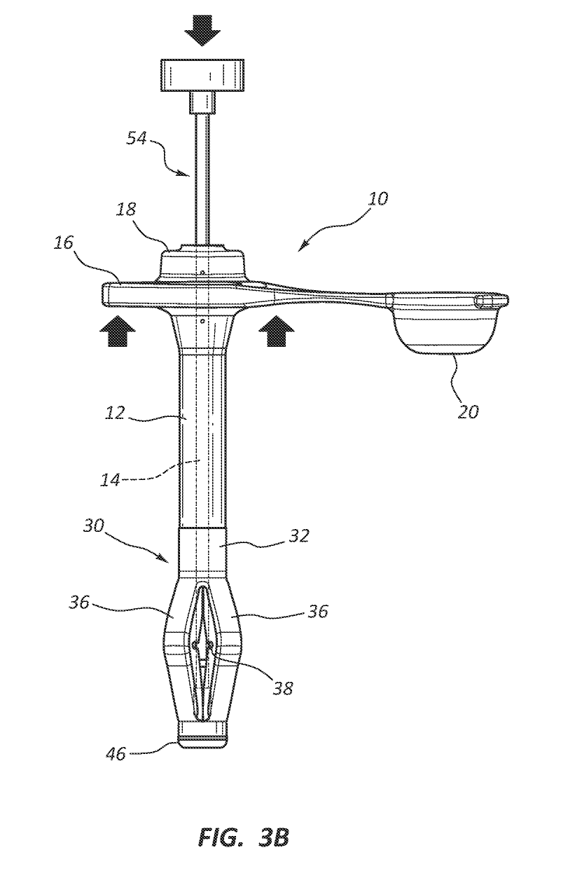

Reference is made to FIGS. 2-3B in describing operation of the internal bolster 30 of the catheter 10, according to one embodiment. FIG. 3A shows that, in its rest state, the internal bolster 30 is configured such that the arms of the array 34 each maintain a folded, radially extended configuration. This configuration is also referred to herein as a deployed configuration and is useful for securing the catheter 10 within a stoma when the catheter has been placed within the body of a patient.

FIG. 3B shows that the internal bolster 30 is longitudinally extendable such that the arms 34 unfold so as to be placed in a substantially parallel, or undeployed, configuration with respect to the longitudinal length of the catheter tube 12. An obturator 54, shown in FIG. 2, can be inserted through the valve assembly 18 and into the lumen 14 of the catheter tube 12 so as to stretch the resilient distal portion 40 of the catheter tube, which in turn longitudinally extends the internal bolster 30 from the folded configuration in FIG. 3A to the configuration shown in FIG. 3B. In one embodiment, and as shown by the force arrows in FIG. 3B, longitudinal extension of the arms 36 of the bolster 30 can be performed manually, such as via thumb pressure on the handle of the obturator 54 and corresponding finger support under the external bolster 16.

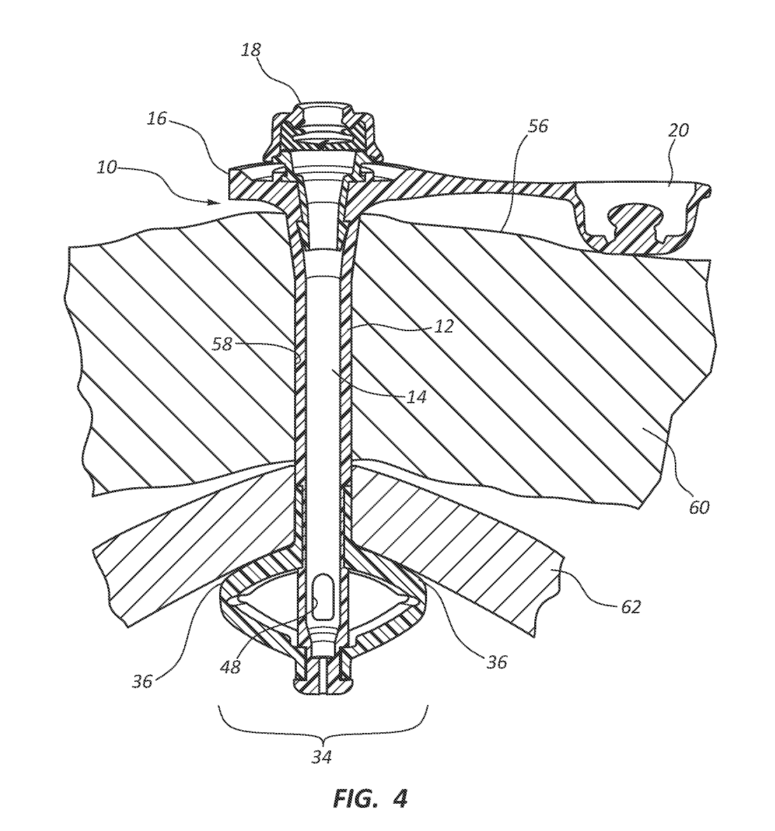

Thus, in one embodiment the internal bolster 30 can be stretched (by the obturator 54 or by another suitable method) to the undeployed configuration of FIG. 3B to insert the catheter 10 into a stoma defined in the body of the patient, such as the stoma 58 shown in FIG. 4, or other suitable body location. After the catheter 10 has been inserted, the obturator 54 can be removed from the catheter tube 12, which enables the resilient distal portion 40 of the catheter tube 12 to urge the internal bolster 30 back to its rest state, wherein the arms 36 of the array 34 return to their folded, radially extended configuration, shown in FIG. 4. In the present example shown in FIG. 4, for instance, the internal bolster 30 is employed to secure a wall 62 of the patient's stomach to adjacent tissue 60 below the patient's skin 56. This in turn enables enteral nutritive liquids to be passed through the catheter tube 12 and into the stomach of the patient. As mentioned, this is but one possible use for the internal boslter of the catheter; indeed the catheter can be employed in other suitable medical and non-medical applications, as appreciated by one skilled in the art. Further, the internal bolsters described herein can be employed in a variety of non-catheter medical devices.

It is thus seen that the distal portion 40 of the catheter tube serves as a resilient member that is centrally disposed in relation to the internal bolster 30 so as to urge the bolster to return from its longitudinally extended configuration (FIG. 3B) to its folded at-rest state wherein the arms are folded in a radially extended configuration (FIG. 4). The folding nature of the arms 36 is also described herein as winged or articulating in movement when changing between the two states described immediately above.

It is further appreciated that a distal portion of the body 32 of the internal bolster 30, or other suitable portion of the bolster or catheter tube 12, can include a radiopaque material 49 (FIG. 3A) so as to enable the internal bolster to be visible during x-ray-based imaging. In one embodiment, the radiopaque material can include metal or tungsten or barium-loaded plastic, for example, to enable the radiopacity.

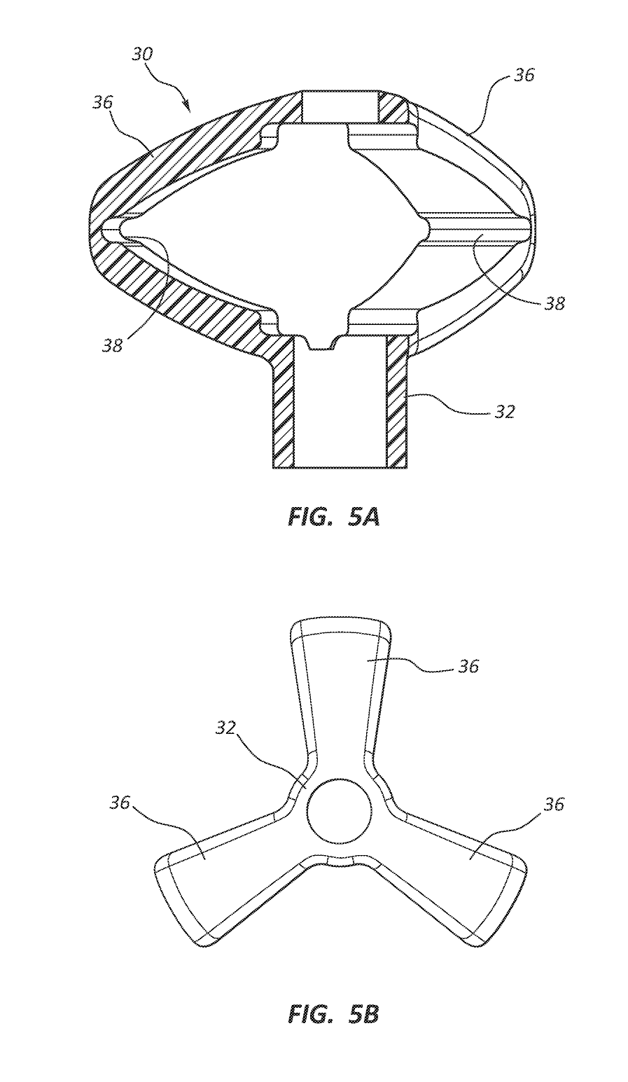

FIGS. 5A and 5B show that the arms 36 of the internal bolster 32 can vary from that described in the previous embodiment. Indeed, as shown in FIGS. 5A and 5B, the internal bolster 30 includes three arms 36 instead of four, as in the previous embodiment. In addition, each arm 36 varies in thickness along its length, best seen in FIG. 5B. More generally, it is appreciated that the number, size, and shape of the arms can vary from what is explicitly shown and described herein.

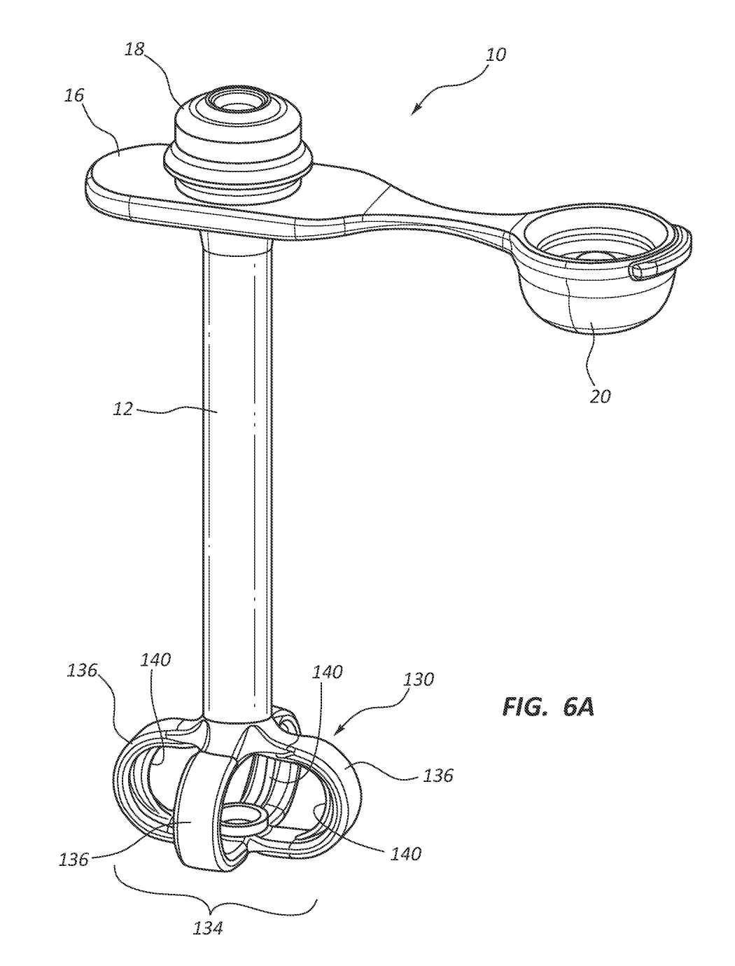

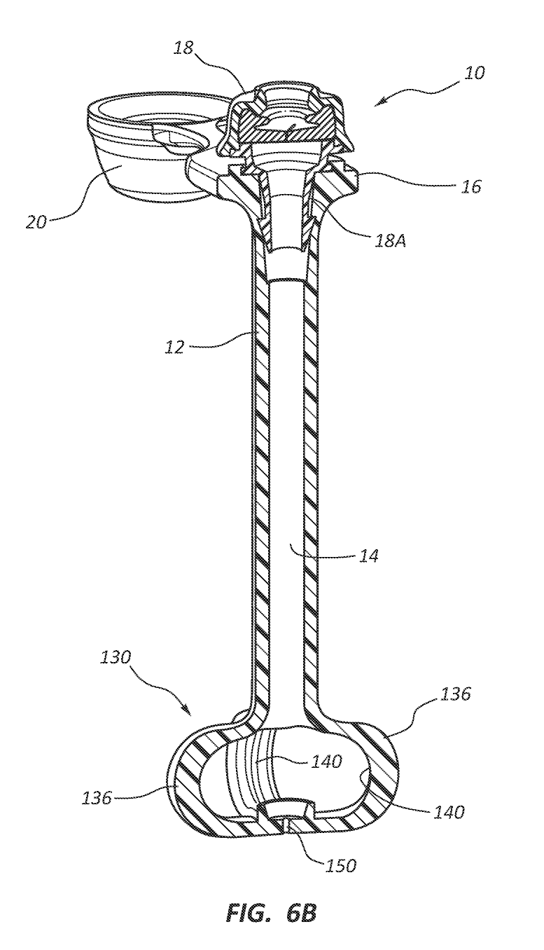

FIGS. 6A and 6B depict details of the catheter 10 and an internal bolster 130 according to another embodiment, wherein the bolster includes an array 134 of arms 136. As shown, each arm 136 is substantially U-shaped, with each terminal end centrally attached to the body of the internal bolster 130 such that the U-shaped arm arcuately extends radially outward to define the perimeter of the bolster. Further, each arm 136 includes a reinforcing rib 140 that extends along the longitudinal length of the arm. The rib 140 of each arm 136 is disposed on an inner surface of the arm and projects radially inward toward the center of the internal bolster 130, though it is appreciated that the rib can take one or more of many forms. Indeed, the size, shape, and number of reinforcing ribs can vary from what is shown and described herein. For instance, two parallel ribs can extend side-by-side along an inner surface of the arm, or ribs can be disposed on both outer and inner arm surfaces. In another embodiment, the rib can be integrated into the internal structure of the arm and can include a material different from that of the arm, such as metal, plastic, etc.

In the present embodiment, the reinforcing ribs 140 assist in increasing the rigidity of each arm 136 such that the internal bolster 130 is effective in preventing inadvertent removal of the catheter 10 from the stoma or other inserted location within the body.

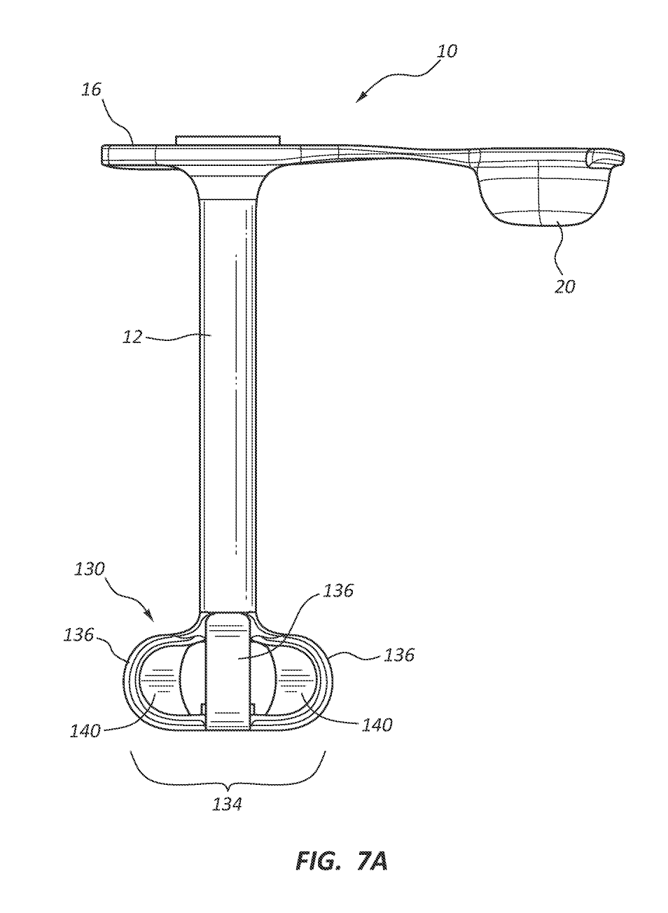

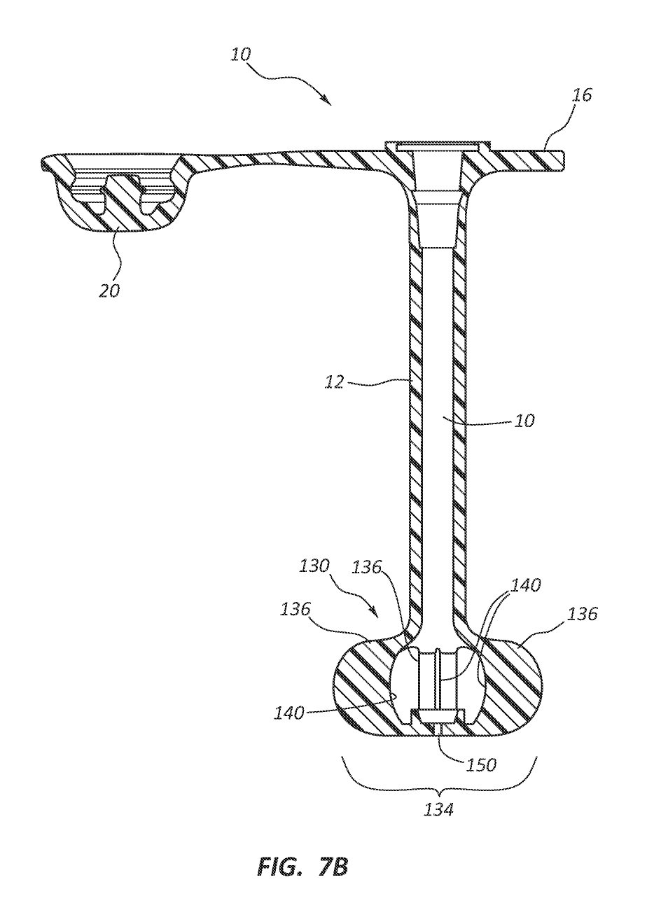

FIGS. 7A and 7B show the internal bolster 130 according to another embodiment, wherein the reinforcing ribs 140 of each arm 136 project radially inward a greater distance relative to the ribs of FIGS. 6A and 6B, thus offering increased rigidity for the arms. These and other variations are therefore contemplated.

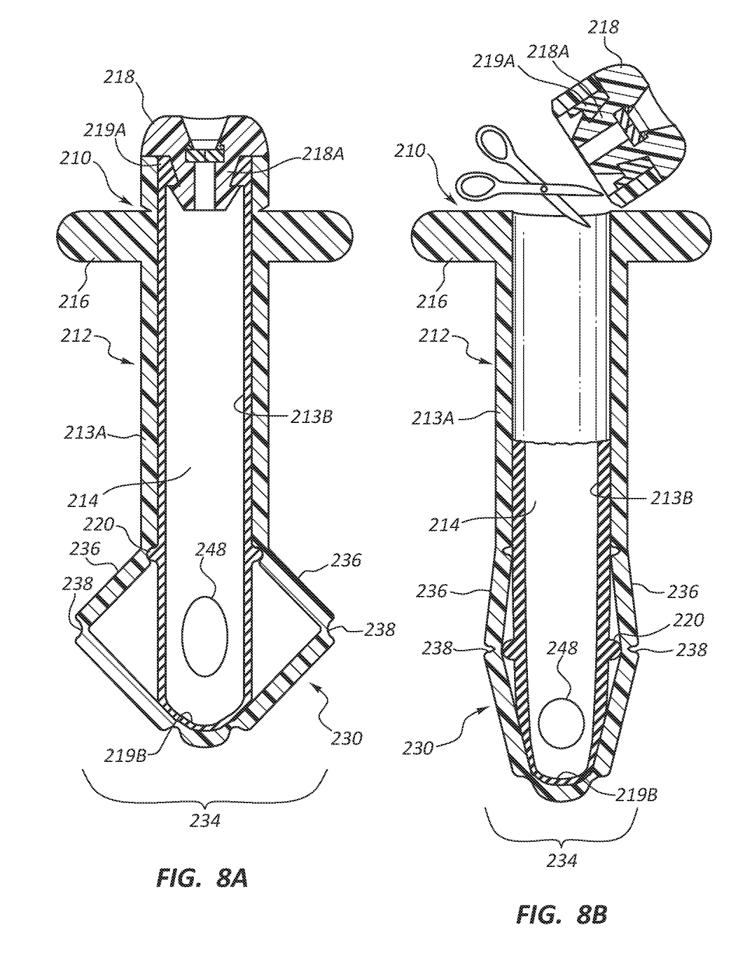

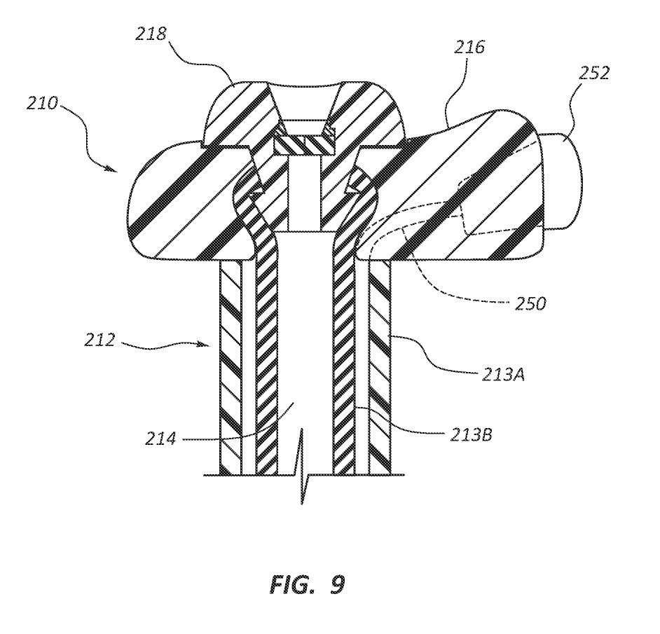

FIGS. 8A and 8B depict details of a catheter 210 according to another embodiment, including an elongate body 212 defined by both an outer tube 213A and an inner tube 213B coaxially received within the outer tube and defining a lumen 214. An external bolster 216 is included, as is a valve assembly 218 with a valve neck 218A that is received into the proximal end of the lumen 214. The proximal ends of the outer tube 213A and inner tube 213B are joined at a proximal connection point 219A, while the distal ends thereof are joined at a distal connection point 219B. In another embodiment, insertion of the valve assembly 218 into the inner tube 213B can be employed to maintain engagement of the inner tube and outer tube 213A (see, e.g., FIG. 9).

A distal portion of the outer tube 213A defines an internal bolster 230, including an array 234 of arms 236, each arm including a living hinge 238 or other suitable hinge. The arms 236 are biased to maintain the partially folded, deployed configuration shown in FIG. 8A when at rest. In the present embodiment, this deployed configuration is achieved by the attachment of the resilient inner tube 213B to the distal end of the outer tube 213A defining the internal bolster 230, which urges the internal bolster 230 into the folded, deployed configuration. As with other embodiments, the internal bolster 230 can be longitudinally extended for insertion/removal of the device by temporarily inserting an obturator through the catheter tube 212. One or more openings 248 are defined in the inner tube 213B to enable fluids to flow out from the catheter tube lumen 214. Also, an annular seal 220 is defined about a portion of the inner tube 213B proximal to the internal bolster 230 to prevent fluids from entering the space between the outer tube 213A and the inner tube.

When removal of the catheter 210 from the body is desired, in one embodiment the catheter tube 212 is cut proximal to the external bolster, which frees the inner tube 213B from engagement with the outer tube 213A. This in turn allows the resilient inner tube 213B to contract, which enables the internal bolster 230 to assume an undeployed, longitudinally extended configuration and be readily removed from the stoma.

FIG. 9 shows that, in one embodiment, a flush lumen 250 and flush valve 252 can be included in the catheter tube 212. A lubricant or suitable fluid can be injected into the flush lumen 250 via the flush valve 252 to reduce friction between and enable relative movement of the outer tube 213A and inner tube 213B. Also, FIG. 9 shows that, in one embodiment, the inner tube 213B can be held in place with respect to the outer tube 213A by insertion of the valve assembly 218 into the lumen 214. Removal of the valve assembly 218 from the lumen 214 causes contraction of the inner tube, thus enabling the internal bolster to extend to its undeployed configuration. These and other bolster configurations and variations are contemplated.

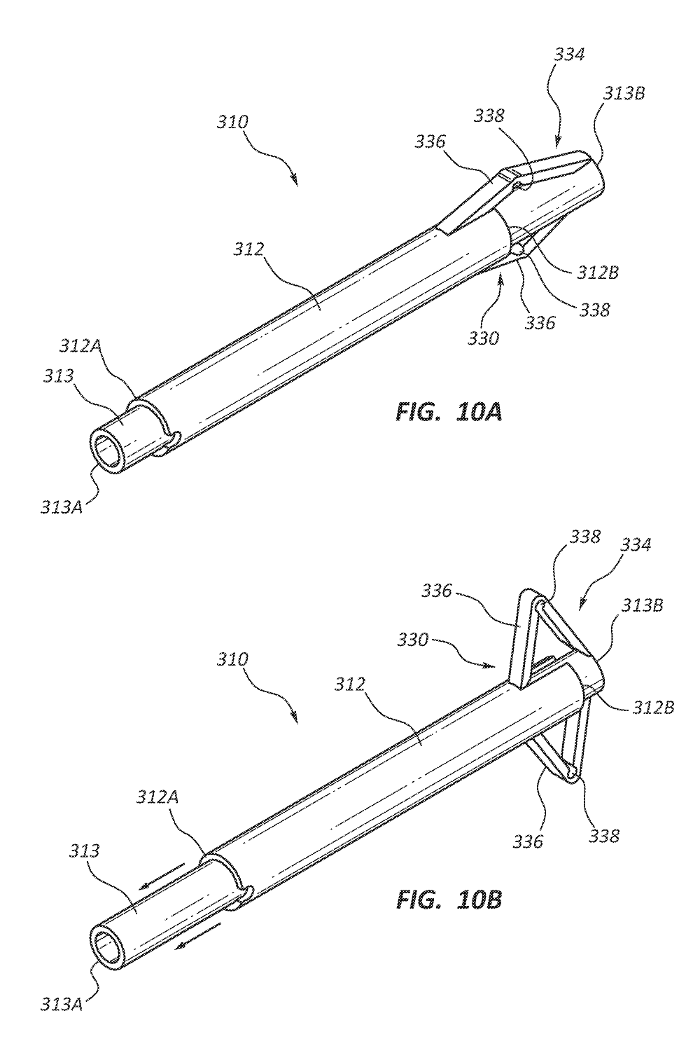

FIGS. 10A and 10B depict a catheter 310 according to another embodiment, wherein the catheter tube is defined by an outer tube 312 that coaxially and slidably receives an inner tube 314. An internal bolster 330 including an array 334 of arms 336, each bendable about a hinge 338, is also included. A proximal end of each arm 336 is attached to a distal portion 312B of the outer tube 312, while a distal end of each arm is attached to a distal portion 314B of the inner tube 314, as seen in FIG. 10A. So configured, the internal bolster 330 is deployed by sliding the inner tube 314 proximally with respect to the outer tube 312. As seen in FIG. 10B, this causes the arms 336 to fold about the hinges 338 and extend radially outward. Correspondingly, distal sliding movement of the inner tube 314 with respect to the outer tube 312 causes the arms 336 to longitudinally extend so as to approach a substantially parallel configuration with respect to the catheter body, as seen in FIG. 10A. In this way, the internal bolster 330 can be selectively deployed.

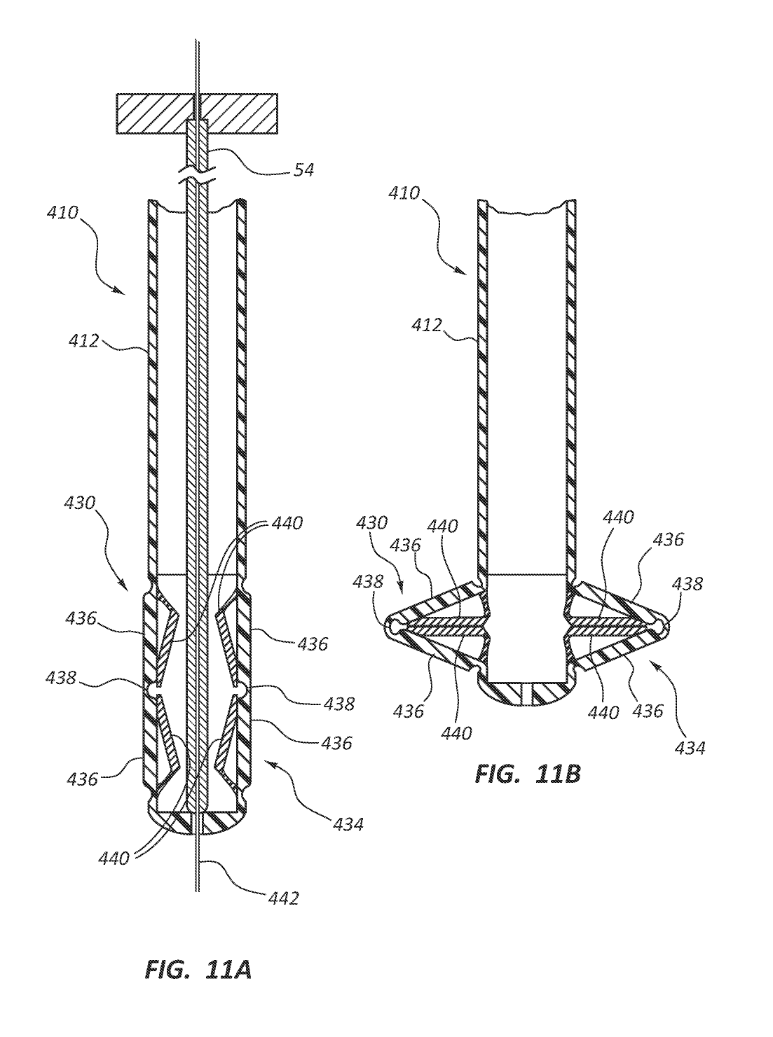

FIGS. 11A and 11B depict a catheter 410 according to another embodiment, including a catheter tube 412 and an internal bolster 430 disposed at the distal end thereof. An array 434 of arms 436, each bendable about a hinge 438, is included in the internal bolster 430. Each arm 436 includes a pair of magnets 440 that are disposed in a spaced-apart arrangement on an inner arm surface on either side of the respective hinge 438, as shown in FIG. 11A. The magnets 440 for each arm 436 are angled with respect to one another and are oriented in their respective magnetic polarities such that the magnets of each pair attract one another.

This magnetic attraction provides a force to bias the internal bolster 430 to a deployed position, as seen in FIG. 11B, wherein the magnets 440 of each arm 436 substantially contact one another. This configuration enables the internal bolster 430 to assist in preventing unintended removal of the catheter 410 from the body of the patient. FIG. 11A shows that an obturator, such as the obturator 54 or other suitable component, can be employed to overcome the magnetic attraction provided by the magnets 440 of each arm 436 and longitudinally extend the arms to an undeployed position, such as for insertion/removal of the catheter 410. A guidewire 442 (FIG. 11A) can be inserted through the catheter 410 to assist with placement thereof. Note that in one embodiment the catheter tube includes silicone, polyurethane, or other suitable material. Also note that a thin coating can cover the magnets 440, in one embodiment. Further, the size, number, type, and placement of the magnets can vary from what is shown and described herein. For instance, though permanent ferromagnets are employed here, in other embodiments, other types of magnets or magnetic elements can be employed.

As mentioned, the internal bolsters depicted and described herein are not limited to use with feeding catheters or feeding devices. Indeed, a variety of medical and other devices that are configured for insertion and anchoring into a body of a patient may benefit from the principles described herein.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A catheter, comprising:
an elongate catheter tube defining at least one lumen;
an internal bolster disposed proximate a distal end of the catheter tube, the internal bolster including a plurality of hinged arms having an un-deployed position wherein each arm is longitudinally extended substantially parallel to the catheter tube, and a deployed position wherein each arm is folded about a hinge so as to radially extend from the catheter tube and provide a deployed bolster configuration; and
a resilient portion included with the catheter tube and disposed centrally with respect to the internal bolster, the resilient portion providing a force urging the internal bolster to the deployed position.

2. The catheter as defined in claim 1, wherein the catheter is configured for insertion into a percutaneous stoma defined in a body of a patient, and wherein the internal bolster is disposed within the body and configured to prevent inadvertent removal of the catheter from the stoma.

3. The catheter as defined in claim 1, wherein the catheter is configured as a feeding tube and wherein the catheter tube includes a lumen opening proximate the internal bolster for providing enteral nutrition to the patient.

4. The catheter as defined in claim 1, further comprising an external bolster proximate a proximal end of the catheter tube, the catheter further including a valve assembly disposed in communication with the catheter tube lumen.

5. The catheter as defined in claim 4, wherein the valve assembly includes a neck portion received into the proximal end of the catheter tube, the valve assembly further including a cap that is removably attached to the valve assembly.

6. The catheter as defined in claim 1, wherein the internal bolster includes a radiopaque portion to enable visualization of the internal bolster using x-ray imaging technology.

7. The catheter as defined in claim 1, wherein the resilient portion is integrally formed with the catheter tube and defines the distal portion of the catheter tube, the lumen extending through the resilient portion.

8. The catheter as defined in claim 7, wherein the catheter tube and resilient portion are integrally formed from silicone, and wherein the internal bolster is changed from the deployed configuration to the undeployed configuration via insertion of an obturator through the catheter tube and resilient portion.

9. The catheter as defined in claim 8, wherein the internal bolster includes four arms and is removably attached to the catheter tube about the resilient portion.

10. The catheter as defined in claim 1, wherein each arm varies in width along a longitudinal length thereof.

11. The catheter as defined in claim 1, wherein the catheter tube includes an inner tube coaxially received within an outer tube, the outer tube defining the internal bolster, the inner tube defining the lumen and the resilient portion.

12. The catheter as defined in claim 11, wherein the inner tube is secured to the outer tube via a valve assembly inserted into catheter tube lumen defined by the inner tube.

13. The catheter tube as defined in claim 12, wherein the inner tube is released from securement with the outer tube by removing the valve.

14. The catheter as defined in claim 11, further comprising a flushing lumen defined between the inner tube and outer tube, wherein liquid is injectable into the flush lumen to reduce friction between the inner tube and outer tube, the flush lumen being in fluid communication with a flush valve included with the catheter.

15. The catheter as defined in claim 14, wherein the inner tube is secured to the outer tube proximate the proximal end of the catheter tube, and wherein the inner tube is released from securement with the outer tube by cutting off a proximal portion of the catheter tube.

16. The catheter as defined in claim 15, wherein releasing the securement of the inner tube with the outer tube enables the internal bolster to change from the deployed configuration to the undeployed configuration.

17. An internal bolster for anchoring a medical device in a percutaneous stoma in a body of a patient, the internal bolster comprising:
a plurality of resilient, looped arms disposed at a distal end of the medical device, each arm extending radially outward from the medical device so as to prevent the medical device from being inadvertently removed from the stoma, each arm including at least one reinforcing rib extending longitudinally along the arm.

18. The internal bolster as defined in claim 17, wherein the medical device is a catheter, wherein the internal bolster is disposed at a distal end of a catheter tube of the catheter, the catheter tube defining at least one lumen.

19. The internal bolster as defined in claim 17, wherein each arm of the internal bolster is attached to a body of the bolster such that a U-shaped portion of each arm extends radially outward from the medical device.

20. The internal bolster as defined in claim 17, wherein the internal bolster includes four arms and wherein the arms can be resiliently deformed by an obturator inserted into the medical device to permit insertion or removal of the medical device with respect to the stoma.

21. The internal bolster as defined in claim 17, wherein the reinforcing rib for each arm is disposed on an inner facing surface so as to extend radially inward.

22. The internal bolster as defined in claim 17, wherein the arms and the reinforcing ribs include silicone.

23. The internal bolster as defined in claim 17, wherein the reinforcing rib of each arm includes a plastic material and is disposed within the arm.

24. The internal bolster as defined in claim 17, wherein a width of each arm varies along the longitudinal length of the arm, and wherein more than one reinforcing rib is included on each arm.

25. A feeding catheter for providing fluid access to a stomach of a patient via a percutaneous stoma, the feeding catheter comprising:
an elongate inner tube defining at least one lumen and coaxially and disposed within an outer tube, the inner tube slidable with respect to the outer tube; and
an internal bolster comprising a plurality of hinged arms, a proximal end of each arm attached to a distal portion of the outer tube, a distal end of each arm attached to a distal portion of the inner tube, wherein proximal movement of the inner tube with respect to the outer tube causes the arms of the internal bolster to fold into an radially extended configuration so as to prevent inadvertent removal of the catheter from the stoma.

26. The feeding catheter as defined in claim 25, wherein distal movement of the inner tube with respect to the outer tube causes arms to change from the folded radially extended configuration to a flattened undeployed configuration so as to permit removal of catheter from stoma.

27. A catheter, comprising:
an elongate catheter tube defining at least one lumen;
an internal bolster disposed proximate a distal end of the catheter tube, the internal bolster including a plurality of hinged arms having an undeployed position wherein each arm is longitudinally extended substantially parallel to the catheter tube, and a deployed position wherein each arm is folded about a hinge so as to radially extend from the catheter tube and provide a deployed bolster configuration; and
a pair of magnetic elements included on each arm and disposed in a spaced-apart relationship about the hinge so as to provide a force urging the arm to the deployed position.
